(19) United States
(12) Patent Application Publication
(10) Pub. No.: US 2026/0259213 A1
(43) Pub. Date: Sep. 3, 2026

(54) PRO-C5 Assay

(71) Applicant: Nordic Bioscience A/S, Herlev (DK)

(72) Inventors: Nicolas Willumsen, Gentofte (DK); Neel I. Nissen, Herlev (DK); Morten Asser Karsdal, København Ø (DK)

(73) Assignee: Nordic Bioscience A/S, Herlev (DK)

(21) Appl. No.: 19/151,183

(22) PCT Filed: Jan. 25, 2024
(86) PCT No.: PCT/EP2024/051828

§ 371 Date: Jul. 25, 2025

(30) Foreign Application Priority Data
Jan. 26, 2023 (GB) 2301141.4

(51) Int. Cl. G01N 33/57525; G01N 33/543; G01N 33/575

(52) U.S. Cl. CPC G01N 33/57525; G01N 33/54306; G01N 33/57585; G01N 2470/04; G01N 2470/10

(57) ABSTRACT

Disclosed herein are methods of immunoassay for detecting and/or monitoring a cancer in a patient by measuring the levels of the N-terminus amino acid sequence TAALGDIMGH (SEQ ID NO: 1) (“PRO-C5”) of the cleaved C-terminal propeptide of the α2 chain of type V collagen in a patient sample.

### FIELD OF THE INVENTION

The present invention relates to methods of immunoassay suitable for detecting and/or monitoring a cancer in a patient.

### BACKGROUND

Pancreatic ductal adenocarcinoma (PDAC) is a devastating disease where most patients present with metastases at diagnosis, resulting in only 20% of the patients being eligible for surgery^{[1-4]}. Additionally, patients with PDAC experience a high degree of treatment resistance. Thus, palliative treatment including chemotherapy is often standard of care^{[2]}. One explanation for the impaired treatment response is the fact that the PDAC tumor microenvironment is extremely fibrotic. Tumor fibrosis causes increased interstitial pressure, which in turn reduces drug delivery^{[5,6]}. In addition, tumor fibrosis has also been shown to inhibit T-cell activity and migration resulting in diminished efficacy of immunotherapy^{[7-11]}. Therefore, there is a high need for novel strategies to overcome tumor fibrosis in the treatment of patients with PDAC and consequently to identify patients with high fibrotic activity.

Tumor fibrosis is characterized by an augmented activity of cancer-associated fibroblasts (CAF), responsible for abnormal extracellular matrix (ECM) remodeling^{[12]}. The CAF-mediated ECM remodeling results in degradation of existing collagen fibers that are replaced by new and more densely packed collagen fibers, creating a stromal barrier surrounding the tumor cells^{[12,13]}. Type I collagen is the most abundant CAF derived fibrillar collagen and a major component of the collagen fibers that play important roles during tumor progression^{[14-19]}. The pathogenic phenotype of CAFs was originally thought to stem from their ability to produce large quantities of collagen, but recent studies focusing on type I collagen have emphasized the importance of the collagen quality and fiber architecture as a central component in tumorigenic capacity^{[14,20]}. Interestingly, the minor fibrillar type V collagen has been suggested to be a key regulator of type I collagen architecture.

Type V collagen has many roles in the healthy ECM, as it can bind to different ECM proteins such as other collagens, TGF-β, elastin and metalloproteinases, thereby modulating cellular behavior^{[21]}. The most abundant isoform of type V collagen consists of two α1 chains and one α2 chain that form a heterotrimer. In healthy tissue, type I and V collagen have a very close relationship, as the two collagens copolymerize into heterotypic fibrils^{[22-24]}. The binding of type V collagen to type I collagen is extremely important for structural integrity. A good example of this is that deficiency of type V collagen results in Ehlers Danlos syndrome, which is characterized by abnormal collagen fibrils^{[25]}. Another study has shown that type V collagen deficient mice (COL5a1−/−) embryos die at day 10.5 and lack type I collagen fibrils. Additionally, heterozygous type V collagen mice (COL5a1+/−) survive but show a 50% decrease in collagen content and fibrillar density^{[22]}. Thus, the binding between type V and I collagen results in the homeostatic fibril structure and tissue architecture. In comparison to type I collagen, type V collagen is expressed in relatively low levels in healthy tissues, and a complete understanding of its role in tumor progression remains unknown^{[25]}. Type V collagen has been shown to be upregulated in some cancers such as colorectal cancer (CRC), gastric cancer and breast cancer as well as being associated with cell proliferation, invasion, metastasis, and angiogenesis^{[24-34]}.

### SUMMARY OF THE INVENTION

The applicant has explored the biomarker potential of measuring the levels of the N-terminus amino acid sequence TAALGDIMGH (SEQ ID NO: 1) (also referred to herein as “PRO-C5”, the “target sequence” or the “PRO-C5 target sequence”) of the cleaved C-terminal propeptide of the α2 chain of type V collagen in the serum of cancer patients. The applicant has demonstrated that the levels of PRO-C5 were significantly elevated in a discovery cohort and a validation cohort of patients with PDAC and in a cohort of patients with various other types of cancer, thus demonstrating the utility of PRO-C5 as a biomarker for cancer.

Accordingly, in a first aspect the present invention provides a method of immunoassay for detecting and/or monitoring a cancer in a patient, the method comprising;

i) contacting a patient sample with a monoclonal antibody that specifically binds to the N-terminus amino acid sequence TAALGDIMGH (SEQ ID NO: 1);
ii) detecting and determining the amount of binding between said monoclonal antibody and peptides in the sample; and
iii) correlating said amount of binding with values associated with normal healthy subjects and/or values associated with known disease severity and/or values obtained from said patient at a previous time point and/or with a predetermined cut-off value.

As used herein the term “N-terminus” refers to an N-terminal peptide sequence at the extremity of a polypeptide, i.e. at the N-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (i.e. a cancer) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. a without cancer); and the term “values associated with known disease severity” means standardised quantities of binding determined by the method described supra for samples from patients known to have disease (i.e. a cancer) of a known severity.

In preferred embodiments, the method may be a method of immunoassay for detecting and/or monitoring a bladder cancer, breast cancer, colorectal cancer, head and neck cancer, kidney cancer, lung cancer, melanoma, ovarian cancer, pancreatic cancer, prostate cancer or stomach cancer. In particular, the method may be a method of immunoassay for detecting and/or monitoring pancreatic ductal adenocarcinoma.

In certain embodiments, the method may be a method of immunoassay for detecting a cancer of a particular severity in a patient. In some embodiments, the method may be a method for detecting a stage of a cancer in a patient. In some embodiments, the method may be a method for detecting a cancer of a level of severity associated with a likely or average overall survival time.

In a preferred embodiment, the monoclonal antibody does not specifically bind to a peptide having the N-terminus amino acid sequence LTAALGDIMGH (SEQ ID NO: 2) (i.e. an elongated version of the PRO-C5 target sequence that, in comparison to the PRO-C5 target sequence, has been extended at its N-terminus by the addition of a lysine residue). Preferably, the ratio of the affinity of said antibody for the PRO-C5 target sequence to the affinity of said antibody for the elongated version of the target sequence is at least 10 to 1, and more preferably is at least 20 to 1 or at least 30 to 1.

In a preferred embodiment, the monoclonal antibody does not specifically bind to a peptide having the N-terminus amino acid sequence AALGDIMGH (SEQ ID NO: 3) (i.e. a truncated version of the PRO-C5 target sequence that, in comparison to the PRO-C5 target sequence, has been truncated by removal of the N-terminus threonine residue). Preferably, the ratio of the affinity of said antibody for the PRO-C5 target sequence to the affinity of said antibody for the truncated version of the target sequence is at least 10 to 1, and more preferably is at least 20 to 1 or at least 30 to 1.

In a preferred embodiment, the monoclonal antibody is raised against a synthetic peptide having the N-terminus amino acid sequence TAALGDIMGH (SEQ ID NO: 1). For example, the monoclonal antibody may be raised by: (a) immunizing a rodent (or other suitable mammal) with a synthetic peptide comprising the N-terminus sequence TAALGDIMGH (SEQ ID NO: 1), which peptide may optionally be linked at its C-terminus to an immunogenic carrier protein (such as keyhole limpet hemocyanin); (b) isolating and cloning a single antibody producing cell; and (c) assaying the resulting monoclonal antibodies to ensure that they have the desired specificity.

In a preferred embodiment, the patient sample is selected from blood, serum or plasma.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

In a second aspect, the present invention provides a method of treating a cancer in a patient in need thereof, the method comprising:
(a) carrying out a method of immunoassay for detecting a cancer in accordance with the first aspect of the present invention on a sample from a patient; and
(b) administering to the patient a therapy for the treatment of said cancer if it is determined in step (a) that the patient has said cancer.

Preferred embodiments of the method in accordance with the second aspect will be apparent from the foregoing discussion of preferred embodiments of the methods according to the first aspect. For example, step (a) may in particular comprise carrying out the method of immunoassay to detect a bladder cancer, breast cancer, colorectal cancer, head and neck cancer, kidney cancer, lung cancer, melanoma, ovarian cancer, pancreatic cancer, prostate cancer or stomach cancer; and/or to detect a cancer of a particular severity.

The therapy may be any therapy suitable for treating the cancer in question. The therapy may for example comprise or consist of one or more surgeries, one or more radiation therapies, one or more medicaments (such as for example one or more chemotherapies, one or more immunotherapies and/or one or more hormonal therapies), or combinations thereof. Medicaments may be formulated for topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, debulking surgeries, palliative surgeries and/or restorative surgeries.

For example, where the cancer is bladder cancer, suitable therapies may comprise one or more of: transurethral resection of bladder tumor (TURBT) with or without intravesical chemotherapy or immunotherapy; radical cystectomy plus neoadjuvant chemotherapy or transurethral resection with chemoradiation or partial cystectomy plus neoadjuvant chemotherapy; cisplatin-based chemotherapy, optionally followed by radical cystectomy or chemoradiation; carboplatin-based chemotherapy; immune checkpoint inhibitors; radical cystectomy; and palliative radiotherapy.

Where the cancer is breast cancer, suitable therapies may for example comprise one or more of: a mastectomy, quadrantectomy or lumpectomy; estrogen receptor blockers (such as tamoxifen); aromatase inhibitors (such as anastrozole or letrozole) that block production of estrogen; CDK inhibitors; one or more chemotherapeutic agents such as a combination of cyclophosphamide, doxorubicin and optionally a taxane (such as docetaxel), or a combination of cyclophosphamide, methotrexate, and fluorouracil; one or more monoclonal antibodies such as trastuzumab and/or pertuzumab; and radiotherapy.

Where the cancer is colorectal cancer, suitable therapies may for example comprise one or more of: endoscopic mucosal resection or endoscopic submucosal dissection; a partial colectomy (or proctocolectomy for rectal lesions); chemotherapy agents such as for example capecitabine, fluorouracil, irinotecan, oxaliplatin or UFT; antiangiogenic drugs such as for example bevacizumab; epidermal growth factor receptor inhibitors, such as for example aflibercept, cetuximab and panitumumab; radiation therapy; immune checkpoint inhibitors; and monoclonal antibodies such as pembrolizumab or dostarlimab.

Where the cancer is head and neck cancer, suitable therapies may for example comprise one or more of: surgery, including but not limited to laser surgery; radiation therapy, including but not limited to 3D conformal radiation therapy, intensity-modulated radiation therapy, particle beam therapy and brachytherapy; one or more chemotherapy agents such as for example, paclitaxel, carboplatin, cetuximab, docetaxel, cisplatin and fluorouracil; photodynamic therapy utilizing amphinex; monoclonal antibodies such as cetuximab, bevacizumab, erlotinib, pembrolizumab or nivolumab; gene therapies such as gendicine; and immune checkpoint inhibitors.

Where the cancer is kidney cancer, suitable therapies may for example comprise one or more of: whole kidney removal or partial removal of the kidney, by surgery; freezing the tumour or treating it with high temperatures; biological therapies such as everolimus, torisel, nexavar, sutent, axitinib, sunitinib, pazopanib, sorafenib, cabozantinib and/or lenvatinib; immunotherapy using interferon and/or interleukin-2; monoclonal antibodies such as nivolumab; immune checkpoint inhibitors; and radiotherapy.

Where the cancer is lung cancer, suitable therapies may for example comprise one or more of: surgery, such as performing a lobectomy, a sublobar excision (wedge resection) or removal of a whole lung (pneumonectomy); radiotherapy, examples of which include radiotherapy given together with chemotherapy, post-operative radiotherapy, brachytherapy (localized radiotherapy) given directly inside the airway, prophylactic cranial irradiation, stereotactic radiation and palliative radiotherapy; chemotherapy using for example one more agents such as cisplatin/carboplatin, etoposide, gemcitabine, paclitaxel, docetaxel, vinorelbine, topotecan, irinotecan and pemetrexed; epidermal growth factor receptor (EGFR) inhibitors drugs such as erlotinib, gefitinib, afatinib, dacomitinib or osimertinib; and immunotherapy, using for example anti PD-L1 monoclonal antibodies such as atezolizumab, nivolumab or pembrolizumab, monoclonal antibodies targeting cytotoxic T-lymphocyte-associated protein 4 (CTLA-4) such as ipilimumab, and/or monoclonal antibodies that targets vascular endothelial growth factor such as bevacizumab.

Where the cancer is melanoma, suitable therapies may for example comprise one or more of: surgical excision of the tumour, and optionally lymph nodes in the area of the tumour; interferon treatment; chemotherapy using agents such as for example dacarbazine; small-molecule targeted therapies using for example BRAF inhibitors (such as vemurafenib and dabrafenib), MEK inhibitors (trametinib), C-Kit inhibitors and/or NRAS inhibitors; immunotherapy using cytokines (e.g. IL-2 and/or IFN-α), immune check point inhibitors (such as for example anti-CTLA-4 monoclonal antibodies such as ipilimumab or tremelimumab, toll-like receptor (TLR) agonists, CD40 agonists, anti-PD-1 antibodies such as pembrolizumab, pidilizumab or nivolumab, LAG-3 inhibitors such as relatlimab, and/or PD-L1 antibodies), and/or adoptive cell transfer (using for example pre-stimulated, modified T cells or dendritic cells); and radiotherapy.

Where the cancer is ovarian cancer, suitable therapies may for example comprise one or more of: removal of one (unilateral oophorectomy) or both ovaries (bilateral oophorectomy), and optionally also the fallopian tubes (salpingectomy), uterus (hysterectomy) and/or the omentum (omentectomy); debulking surgery; chemotherapy (including neoadjuvant or adjuvant chemotherapy) using agents such as paclitaxel, cisplatin, topotecan, doxorubicin, epirubicin, gemcitabine, carboplatin, docetaxel, vincristine, dactinomycin, etoposide, cyclophosphamide, oxaliplatin or combinations thereof; radiotherapy; hormonal therapy; and immunotherapy, such as for example the antibody drug bevacizumab.

For example where the cancer is a pancreatic cancer, such as for example PDAC, suitable therapies may for example comprise one or more of: surgical resection, such as for example tumor resection, the Whipple procedure, total pancreatectomy or distal pancreatectomy; radiotherapy, examples of which include but are not limited to radiotherapy given together with chemotherapy, post-operative radiotherapy, brachytherapy (localized radiotherapy), stereotactic radiation and palliative radiotherapy; chemotherapy using for example one more agents such as gemcitabine, 5-FU, erlotinib, FOLFIRINOX, nab-paclitaxel or combinations thereof; the somatostatin analog class of medications; lanreotide; targeted therapy using everolimus or sunitinib; nuclear medicine therapy with radiolabeled peptides or hormones such as iobenguane; and techniques such as radiofrequency ablation (RFA), cryoablation, or hepatic artery embolization.

Where the cancer is prostate cancer, suitable therapies may for example comprise one or more of: radiotherapy; chemotherapy using chemotherapeutic agents, such as for example docetaxel, cabazitaxel, docetaxel, thalidomide, and combinations thereof; immunotherapy, such as for example the monoclonal antibody bevacizumab; hormonal therapies such as for example abiraterone and enzalutamide; external beam radiation therapy; particle therapy; high-intensity focused ultrasound; cryotherapy; and surgical procedures such as for example a radical prostatectomy.

Where the cancer is stomach cancer, suitable therapies may for example comprise one or more of: surgical procedures such as for example an endoscopic mucosal resection. endoscopic submucosal dissection or gastrectomy; chemotherapy using agents such as fluorouracil, capecitabine, BCNU, methyl-CCNU, and doxorubicin, mitomycin C, cisplatin, Taxotere or combinations thereof; targeted therapy using epidermal growth factor receptor 2 inhibitors, such as trastuzumab; and radiotherapy.

### FIGURES

FIG. 1: Type V collagen regulation of type I collagen architecture. Collagen fibers consist of collagen fibrils, which are made up by self-assembled collagen microfibrils. (A) Type V collagen is incorporated into the type I collagen microfibril structure where, during homeostasis, it regulates structural integrity of type I collagen. (B) Type V collagen deficiency (low COLV) results in abnormal type I collagen fibrils and the well-known Ehlers Danlos syndrome. Augmented type V collagen expression (high COLV) results in more condensed and linearized type I collagen fibrils and dense fibers characteristic of tumor fibrosis. COL I: type I collagen. COL V: type V collagen.

FIG. 2: PRO-C5 is elevated in serum from patients in the PDAC discovery cohort. (A) Individual serum PRO-C5 levels in patients with PDAC (n=33), chronic pancreatitis (n=12) and healthy controls (n=20). (B) Individual serum PRO-C5 levels in patients with either stage I, II, III and IV PDAC. The association between PRO-C5 levels and PDAC stages was analyzed using the least square regression method. ***p<0.001.

FIG. 3: High levels of serum PRO-C5 associates with poor overall survival (OS) in the discovery cohort. A Kaplan-Meier survival plot showing the association between overall survival and levels of PRO-C5. Patients were stratified into quartiles i.e., Q1 containing patients with the lowest levels of PRO-C5 and Q4 containing patients with the highest levels of PRO-C5. Hazard ratios (HR), 95% confidence intervals (CI) and log-rank test are shown.

FIG. 4: High levels of serum PRO-C5 associates with poor OS in the validation cohort. (A) Individual serum PRO-C5 levels in patients with either stage I, II, III and IV PDAC. The association between PRO-C5 levels and PDAC stages was analyzed using the least square regression method. (B) A Kaplan-Meier survival plot showing the association between overall survival and levels of PRO-C5. Patients were stratified into low (Q1-Q3) and high (Q4) PRO-C5 serum levels. Hazard ratios (HR), 95% confidence intervals (CI) and log-rank test are shown.

FIG. 5: High levels of serum PRO-C5 associates with poor OS in stage II, III and IV PDAC in the validation cohort. A Kaplan-Meier survival plot showing the association between overall survival and levels of PRO-C5 in stage II (A), stage III (B) and stage IV (C). In all plots, patients were stratified into low (Q1-Q3) and high (Q4) PRO-C5 serum levels. Hazard ratios (HR), 95% confidence intervals (CI) and log-rank test are shown.

FIG. 6: PRO-C5 is elevated in serum from patients with different types of cancer. Individual serum PRO-C5 levels in patients with various stages of either bladder cancer (n=20), breast cancer (n=20), colorectal cancer (n=20), head and neck cancer (n=20), kidney cancer (n=20), lung cancer (n=20), malignant melanoma (n=20), ovarian cancer (n=20), PDAC (n=20), prostate cancer (n=20) or stomach cancer (n=20) and compared to healthy controls (n=33) are shown. Ns: non-significant, *p<0.05, ***p<0.001, ****p<0.0001.

FIG. 7: Specificity of the PRO-C5 assay. Reactivity towards the standard peptide, an elongated peptide, and a non-sense peptide is shown.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Patient Demographics

Cohort 1, the PDAC discovery cohort, consisted of 19 age and sex matched healthy controls, 12 patients with pancreatitis and 33 patients with PDAC (stage I-IV). Cohort 2, the PDAC validation cohort, consisted of 800 patients with PDAC (stage I-VI). Serum samples from patients with pancreatitis and PDAC, in cohort 1 and 2, were from the Danish BIOPAC study “Biomarkers in patients with pancreatic cancer (BIOPAC)—can they provide new information of the disease and improve diagnosis and prognosis of the patients” (ClinicalTrials.gov ID: NCT03311776). The study has been explained elsewhere^{[35-37]}. The study was carried out in accordance with the Danish Regional Committee on Health Research Ethics. The BIOPAC protocol was approved by the Danish Regional Committee on Health Research Ethics (VEK ref. KA-20060113; and the retrospective protocol VEK H-17039022) and the Data Protection Agency (j.nr. 2006-41-6848, 2012-58-0004, HGH-2015-027; I-Suite j. nr. 03960; and PACTIUS P-2020-834). All subjects gave written informed consent in accordance with the Declaration of Helsinki, version 8. Serum samples and clinical data were collected prospectively. The patients were followed until September 2022 or death, whichever came first. Serum samples were measured blinded without information of the clinical characteristics. Clinical data included age, sex, stage (American Joint commission on cancer, eight edition), number of metastatic sites, liver metastasis, body mass index (BMI diabetes, tobacco use, alcohol use, carbohydrate antigen 19-9 (CA19-9), performance status (PS), The Charlson age comorbidity index (CACI) and overall survival (OS). Cohort 3 consisted of 33 age and sex matched healthy controls and 220 patients with different types of cancers; bladder cancer (n=20), breast cancer (n=20), colorectal cancer (n=20), head and neck cancer (n=20), kidney cancer (n=20), lung cancer (n=20), malignant melanoma (n=20), ovarian cancer (n=20), PDAC (n=20), prostate cancer (n=20), stomach cancer (n=20). Cancer samples from cohort 3 were obtained from the commercial vendor Proteogenex (CA, USA). Healthy controls in cohort 1 and 3 were obtained from Valley BioMedical (VA, USA). Appropriate Institutional Review Board/Independent Ethical Committee approved sample collection and all subjects filed for informed consent. Patient demographics for cohorts 1 and 2 are shown in Table 1 and patient demographics for cohort 3 are shown in Table 2.

TABLE 1
Patient demographics for Cohort 1 and Cohort 2

| Discovery cohort | | Validation cohort |
|---|---|---|
| (Cohort 1) | | (Cohort 2) |
| Clinical variables | Study | |
| (healthy controls) | population | |
| | (n = 20) | |
| Age, (years) | | |

| | | |
|---|---|---|
| Median (min, max) | 58 | (45-72) |
| Gender, n (%) | | |
| Male | 10 | (50%) |
| Female | 10 | (50%) |

| | |
|---|---|
| Clinical variables | Study |
| (chronic pancreatitis) | population |
| | (n = 12) |

| | | |
|---|---|---|
| Age, (years) | | |
| Median (min, max) | 60 | (49-79) |
| Gender, n (%) | | |
| Male | 10 | (83%) |
| Female | 2 | (17%) |

| | Study | | Study | |
|---|---|---|---|---|
| Clinical variables | population | | population | |
| (PDAC) | (n = 33) | | (n = 800) | |
| Age, (years) | | | | |
| Median (min, max) | 69 | (52-79) | 67 | (37-89) |
| Gender, n (%) | | | | |
| Male | 17 | (%) | 428 | (%) |
| Female | 16 | (%) | 372 | (%) |
| Number of metastatic | | | | |
| sites, n (%) | | | | |
| 0 site | 15 | (%) | 364 | (%) |
| ≥1 site | 18 | (%) | 436 | (%) |
| Liver metastasis | | | Liver metastasis | |
| (of all patients | | | (of all patients | |
| with metastasis, | | | with metastasis, | |
| n = 18), n (%) | | | n = 436), n (%) | |
| Yes | 17 | (%) | 324 | (%) |
| No | 1 | (%) | 112 | (%) |
| BMI | | | | |
| Median (min, max) | 23 | (16-31) | 23 | (14-39) |
| Stage | | | | |
| 1 | 1 | (%) | 15 | (%) |
| 2 | 7 | (%) | 119 | (%) |
| 3 | 7 | (%) | 227 | (%) |
| 4 | 18 | (%) | 435 | (%) |
| Unknown | 0 | | 4 | (%) |
| Diabetes | | | | |
| Yes | 8 | (%) | 194 | (%) |
| No | 25 | (%) | 597 | (%) |
| Unknown | 0 | | 9 | (%) |
| Tobacco | | | | |
| Ever | 21 | (%) | 485 | (%) |
| Never | 12 | (%) | 244 | (%) |
| Unknown | 0 | | 71 | (%) |
| Alcohol | | | | |
| <DHAR | 26 | (%) | 551 | (%) |
| >DHAR | 7 | (%) | 176 | (%) |
| Unknown | 0 | | 73 | (%) |
| CA19-9 (U/mL) | | | | |
| ≤median (≤483 U/mL) | 15 | (%) | 386 | (%) |
| >median (>483 U/mL) | 18 | (%) | 388 | (%) |
| Unknown | 0 | | 26 | (%) |
| Performance status, | | | | |
| n (%) | | | | |
| 0 | 13 | (%) | 286 | (%) |
| 1 | 12 | (%) | 335 | (%) |
| 2 | 3 | (%) | 90 | (%) |
| 3 | 0 | | 4 | (%) |
| Unknown | 5 | (%) | 85 | (%) |
| The Charlson age | | | | |
| comorbidity index | | | | |

| | | | |
|---|---|---|---|
| <4 | n/a | 679 | (%) |
| ≥4 | n/a | 108 | (%) |
| Unknown | n/a | 13 | (%) |

TABLE 2
Patient demographics for Cohort 3

| | |
|---|---|
| Healthy controls | Patients with |
| (n = 33) | cancer (n = 220) |

| | | | | |
|---|---|---|---|---|
| Age (years) | | | | |
| Median (Min; Max) | 57.0 | (49.0; 69.0) | 61.0 | (30.0; 87.0) |
| Sex | | | | |
| Male | 21 | (64%) | 119 | (54%) |
| Female | 12 | (36%) | 101 | (46%) |
| Diagnosis | | | | |
| Bladder cancer | | | 20 | (9%) |
| Breast cancer | | | 20 | (9%) |
| Colorectal | | | 20 | (9%) |
| cancer | | | | |
| Head & neck | | | 20 | (9%) |
| cancer | | | | |
| Renal cancer | | | 20 | (9%) |
| Lung cancer | | | 20 | (9%) |
| Melanoma | | | 20 | (9%) |
| Ovarian cancer | | | 20 | (9%) |
| Pancreatic | | | 20 | (9%) |
| cancer | | | | |
| Prostate cancer | | | 20 | (9%) |
| Gastric cancer | | | 20 | (9%) |
| Stages | | | | |
| I | | | 7 | (3%) |
| II | | | 46 | (21%) |
| III | | | 93 | (42%) |
| IV | | | 74 | (34%) |

### Generation of Monoclonal Antibodies

PRO-C5 monoclonal antibodies, i.e. monoclonal antibodies specific for the PRO-C5 target sequence (TAALGDIMGH (SEQ ID NO: 1)) located at the start of the N-terminus of cleaved C-terminal propeptide of the α2 chain of type V collagen, were generated as described previously.

Briefly, six 4-6-week-old Balb/C mice were immunized subcutaneously in the abdomen with 200 μL emulsified antigen and 50 μg immunogenic peptide (TAALGDIMGH-GGC-OVA (SEQ ID NO: 4)) using Freund's incomplete adjuvant. Immunizations were performed at 2-week intervals until stable titer levels were obtained. At each bleeding, the serum antibody titer was measured and the mice with the highest antibody titer and best reactivity toward serum and urine were selected for fusion. The selected mice were boosted intravenously with 50 μg immunogenic peptide in 100 μL 0.9% sodium chloride solution 3 days before isolation of the spleen for cell fusion.

To produce hybridoma cells, the mouse spleen cells were fused with SP2/0 myeloma cells as described by Gefter et al. The hybridoma cells were cloned using a limiting dilution method and transferred into 96-well microtiter plates for further growth. Standard limited dilution was used to promote monoclonal growth. Supernatants were screened using an indirect ELISA, in which the biotinylated peptide TAALGDIMGH-GGC-Biotin (SEQ ID NO: 5) was used as a catcher peptide on streptavidin-coated microtiter plates.

Native reactivity and peptide binding of the monoclonal antibodies in human serum and urine, and in rat serum and urine, was evaluated using a preliminary ELISA with 10 ng/ml biotinylated peptide coater on a streptavidin coated microtiter plate and the supernatant from the growing monoclonal hybridoma. Clone specificity was tested against the standard peptide (TAALGDIMGH (SEQ ID NO: 1)) and a nonsense peptide. Isotyping of the monoclonal antibodies was performed using the Clonotyping System-HRP kit, cat. 5300-05 (Southern Biotech, Birmingham, AL). The selected clones were purified using Protein G columns according to the manufacturer's instructions and dialyzed (GE Healthcare Life Science, Little Chalfont, Buckinghamshire, UK).

### Antibody Specificity

Specificity of the PRO-C5 monoclonal antibody was calculated as percentage of signal inhibition by two-fold diluted standard peptide (TAALGDIMGH (SEQ ID NO: 1)), elongated peptide (LTAALGDIMGH (SEQ ID NO: 2)), and non-sense peptide.

### Assessment of PRO-C5 Levels in Human Serum

Serum levels of PRO-C5 were measured using the ELISA based PRO-C5 assay, according to manufactures instruction's (Nordic Bioscience A/S, Denmark). The technical details of this assay have previously been described^{[38, 39]}.

Briefly, a 96-well streptavidin plate (Roche Diagnostics, Basel, Switzerland) was coated with 3 ng of the biotinylated synthetic peptide, TAALGDIMGH-GGC-Biotin (SEQ ID NO: 5), diluted in coater buffer (25 mM PBS, 1% BSA, 0.1% Tween-20, pH 7.4 and 0.36% Bronidox) and incubated for 30 minutes at 20° C. 20 μL of the peptide calibrator (TAALGDIMGH (SEQ ID NO: 1)) or sample were added to appropriate wells, followed by 100 μL of 15 ng horseradish peroxidase (HRP) (Innovabioscience, Babraham, Cambridge, UK). Conjugated PRO-C5 monoclonal antibodies were diluted in the same buffer that was used for coating, and incubated for 1 hour at 20° C. Finally, 100 μL tetramethylbenzinidine (TMB) (Kem-En-Tec cat. 438OH, Taastrup, Denmark) was added, and the plate was incubated for a further 15 minutes at 20° C. in the dark. All the above incubation steps included shaking at 300 rpm. After each incubation step the plate was washed five times in washing buffer (20 mM Tris, 50 mM NaCl, pH 7.2). The TMB reaction was stopped by adding 100 μL of stopping solution (1% HCl) and measured at 450 nm with 650 nm as the reference. A calibration curve was plotted using a 4-parametric mathematical fit model with a starting concentration of 200 ng for the standard peptide (TAALGDIMGH (SEQ ID NO: 1)) following a 2-fold dilution.

### Statistics

Biomarker results were reported in accordance with the REMARK (reporting recommendations for tumor marker prognostic study) guidelines^{[40]}.

A Kruskal-Wallis multiple comparison test was used to test the difference between PRO-C5 serum levels in healthy controls, patients with pancreatitis and patients with PDAC (cohort 1) as well as for PRO-C5 serum levels in healthy controls and in 11 different cancer indications (cohort 3). A least squared regression analysis was used to assess associations with PRO-C5 serum levels and stages of PDAC in cohort 1 and cohort 2. Kaplan-Meier curves were used to assess the association between high and low serum levels of PRO-C5 and OS. In cohort 1, patients were stratified into four quartiles according to PRO-C5 serum levels (Q1, Q2, Q3 and Q4). In cohort 2, patients were stratified into two groups, one containing quartiles Q1-Q3 and the other containing quartile Q4. When exploring the association between PRO-C5 serum levels and OS in specific stages of PDAC, patients were also stratified into two groups (Q1-Q3 vs. Q4). In cohort 1 a univariate Cox proportional-hazard regression model was used to calculate the hazard ratios (HR) with 95% confidence interval (Cl) for short OS per PRO-C5 biomarker levels (Q4, or Q3 or Q2 vs. Q1). In cohort 2, a univariate Cox proportional-hazard regression model was used to calculate the hazard ratios (HR) with 95% confidence interval (Cl) for short OS per PRO-C5 biomarker levels (continuous and >Q1-Q3 vs ≤Q4) and clinical covariates: age (continuous), gender (female vs male), number of metastatic sites (≥1 vs 0), liver metastasis (yes vs no), BMI (continuous), stage (continuous), diabetes (yes vs no), tobacco use (ever vs never), alcohol use (below and above the Danish Health Authority recommendations [DHAR]), CA19-9 (>median vs ≤median [median=483 U/mL]), PS (1+2+3 VS 0) and CACI (≥4 vs <4). In addition, in cohort 2, a multivariate Cox proportional-hazard regression models including PRO-C5 (continuous and >Q1-Q3 vs <04), age, metastatic sites (≥1 vs 0), liver metastasis (yes vs no), stage (continuous), CA19-9 (>median vs ≤median [median=483 U/mL]), PS (1+2+3 vs 0) and CACI (≥4 vs <4) was used to evaluate potential independent prognostic value of the PRO-C5 biomarker for predicting mortality risk. When the model was used for patients in individual stages only age, CA19-9 (>median vs ≤median [median=483 U/mL]), PS (1+2+3 vs 0) and CACI (≥4 vs <4) were included for stage II and III. For stage IV, age, CA19-9 (>median vs ≤median [median=483 U/mL]), PS (1+2+3 vs 0), CACI (≥4 vs <4), number of metastatic sites (≥1 vs 0) and liver metastasis (yes vs no) were included. P value of P<. 05 was considered statistically significant. Graph design and statistical analyses were performed using GraphPad Prism Version 9 (GraphPad Software, Inc.) and MedCalc version 19.3 (Medcalc Software).

### Results

### Antibody Specificity

To evaluate the specificity of the PRO-C5 monoclonal antibody and assay, the antibody was tested against the standard peptide, elongated peptide and non-sense standard peptide, and showed no reactivity towards the elongated peptide or non-sense standard peptide, demonstrating the specificity of the antibody and assay for the PRO-C5 target sequence (FIG. 7).

PRO-C5 is Elevated in Patients with PDAC and Associates with Poor OS-Discovery Cohort

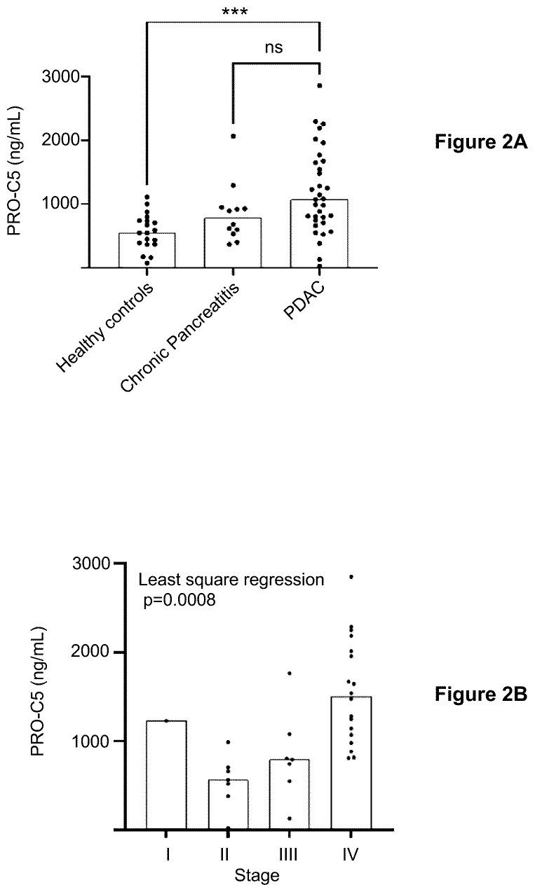

The PDAC discovery cohort (cohort 1) included 19 healthy controls, 12 patients with pancreatitis and 33 patients with PDAC (stage I-IV) (Table 1). PRO-C5 levels were significantly elevated in serum from patients with PDAC compared to healthy controls (PDAC: 1071.3 ng/mL vs. healthy controls: 549.5 ng/mL, p<0.001). PRO-C5 levels were not significantly increased in patients with pancreatitis (pancreatitis: 786.4 ng/ml) compared to healthy controls and in patients with PDAC compared to pancreatitis. However, there was a trend showing an increase in PRO-C5 from healthy controls to pancreatitis to PDAC (FIG. 2A). When stratifying patients with PDAC according to disease stage (stage I-IV), there was a significant correlation between serum PRO-C5 levels and stage of disease (least squares regression: p=0.0005) (FIG. 2B).

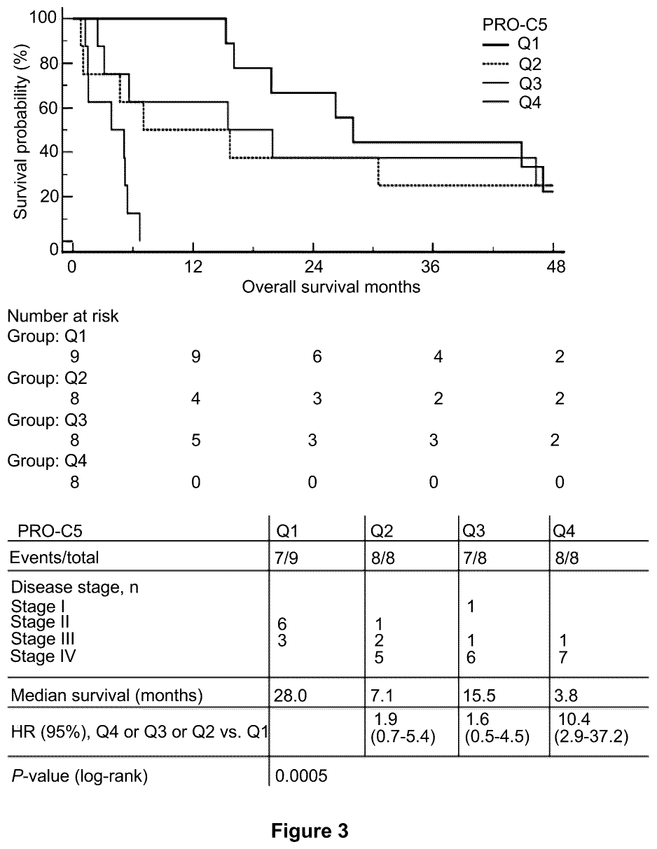

To investigate the association between PRO-C5 serum levels and OS, the prognostic potential of PRO-C5 was assessed by Kaplan-Meier curves and univariate Cox proportional-hazard model. Patients were stratified into quartiles i.e., Q1 containing those patients with the lowest levels of PRO-C5 and Q4 containing the patients with the highest levels of PRO-C5. Patients in Q1 had a median OS of 28.0 months, patients in Q2 had a median OS of 7.1 months, patients in Q3 had a median OS of 15.5 months and patients in Q4 had a median OS of 3.8 months (log-rank, p=0.0005). Thus, the difference in median OS between Q4 and Q1 were more than two years (24.2 months) (FIG. 3). In support, Univariate Cox proportional-hazard model showed that patients in Q4 had 940% increased risk of mortality than patients in Q1 (Q4 vs. Q1: HR 95% CI: 10.4 (2.9-37.2), p=0.0003).

PRO-C5 is Elevated in Patients with PDAC and Associates with Poor OS-Validation Cohort

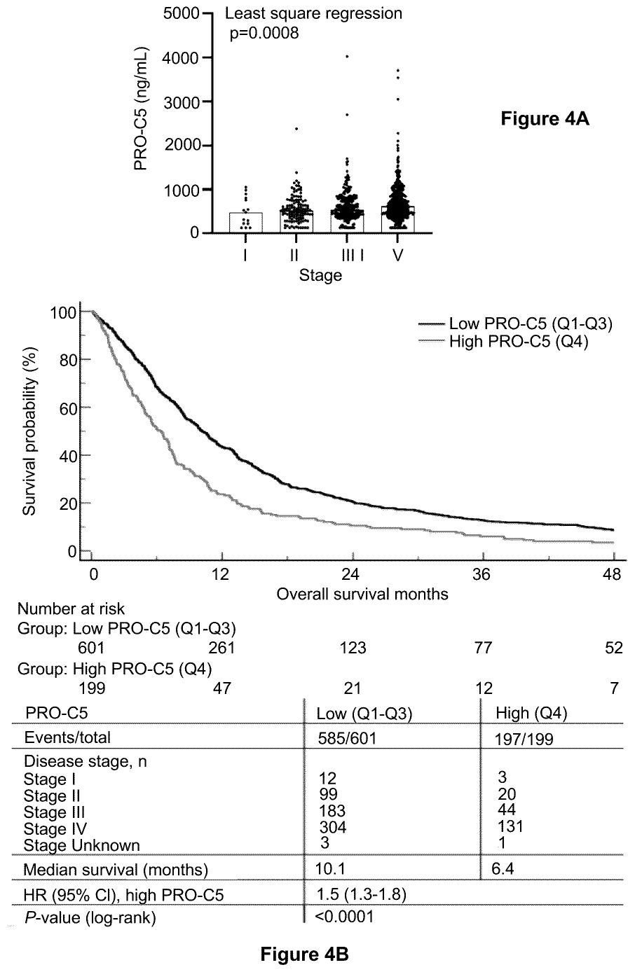

To validate the prognostic potential of PRO-C5 in the PDAC discovery cohort, PRO-C5 levels were measured in serum from a PDAC validation cohort (BIOPAC, cohort 2) containing 800 patients with PDAC (stage I-IV) (Table 1). Overall, the findings from the discovery cohort were confirmed. When stratifying patients into stages (stage I-IV), there was a significant correlation between serum PRO-C5 levels and stage of disease (least squares regression: p=0.0008) (FIG. 4A). When evaluating the association between PRO-C5 serum levels and OS in det PDAC validation cohort, patients were stratified into two groups based on the results from the discovery cohort: one containing patients with the lower levels of serum PRO-C5 (Q1-Q3), and another containing patients with the highest levels of serum PRO-C5 (Q4). The patients with lower PRO-C5 levels had a median OS of 10.1 months, whereas patients with the highest PRO-C5 levels had a median OS of 6.4 months (log-rank p<0.0001) (FIG. 4B). In addition, univariate Cox proportional-hazard modelling showed that the patients with highest levels of PRO-C5 had 50% increased risk of mortality as compared to patients with lower levels of PRO-C5 (High PRO-C5 vs. low PRO-C5: HR 95% CI: 1.5 (1.3-1.8), p<0.0001) (FIG. 4B and Table 3). To evaluate if the association of OS and PRO-C5 was independent of clinical covariates, a multivariate Cox proportional-hazard model including age, number of metastatic sites, liver metastasis, stage, CA19-9, PS and CACI was performed. The model showed that the prognostic value of PRO-C5 remained statistically significant when adjusting for clinical covariates (HR 95% CI: 1.4 (1.2-1.6), p=0.0002) (Table 3), indicating that PRO-C5 is a risk factor independent of other common risk factors.

TABLE 3
Uni- and multivariate cox proportional-hazard regression
models in patients with PDAC (n = 800, stage I-IV).

| | | Cox proportional-hazard | | | |
|---|---|---|---|---|---|
| | | regression models | | | |
| | | Univariate | | Multivariate | |
| | | HR | | HR | |
| Variables | | (95% CI) | P-value | (95% CI) | P-value |
| PRO-C5 | Continuous | 1.0 | <0.0001* | — | — |
| | | (1.0-1.0) | | | |
| PRO-C5 | Q4 vs. | 1.5 | <0.0001* | 1.4 | 0.0002* |
| | Q1-Q3 | (1.3-1.8) | | (1.2-1.6) | |
| Age | Continuous | 1.0 | 0.0356 | 1.0 | 0.3723 |
| | | (1.0-1.0) | | (1.0-1.0) | |
| Sex | Female vs. | 1.0 | 0.6569 | — | — |
| | male | (0.8-1.1) | | | |
| No. of | ≥1 vs. 0 | 2.6 | <0.0001* | 1.6 | 0.001* |
| metastatic | | (2.5-3.0) | | (1.3-2.0) | |
| sites | | | | | |
| Liver | Yes vs. No | 2.3 | <0.0001* | 1.2 | 0.1647 |
| metastasis | | (2.0-2.7) | | (0.9-1.4) | |
| BMI | Continuous | 1.0 | 0.2715 | — | — |
| | | (1.0-1.0) | | | |
| Stage | Continuous | 1.3 | <0.0001* | 1.2 | <0.0001* |
| | | (1.3-1.4) | | (1.1-1.3) | |
| Diabetes | Yes vs. no | 1.1 | 0.3616 | — | — |
| | | (0.9-1.3) | | | |
| Alcohol | >DHAR VS. | 1.0 | 0.8814 | — | — |
| | <DHAR | (0.8-1.3) | | | |
| CA19-9 | >median vs. | 2.0 | <0.0001* | 1.5 | <0.0001* |
| | <median | (1.7-2.3) | | (1.3-1.8) | |
| PS | 1 + 2 + 3 | 1.5 | <0.0001* | 1.4 | <0.0001* |
| | vs. 0 | (1.3-1.8) | | (1.2-1.6) | |
| CACI | High (≥4 | 1.3 | 0.0315 | 1.2 | 0.3604 |
| | vs. <4) | (1.0-1.5) | | (0.9-1.4) | |

Abbreviations: BMI, body mass index; CACI, the Charlson age comorbidity index; CA 19-9, carcinoembryonic antigen 19-9; DHAR, Danish Health Authority recommendations; No, Number; and PS: performance status.
Hazard ratios (HR), 95% confidence intervals (CI).
*Significant p-value.

The association between high serum PRO-C5 levels and OS in patients stratified into specific stages of PDAC was further assessed. Patient in disease stage I were not analyzed due to low n (n=15).

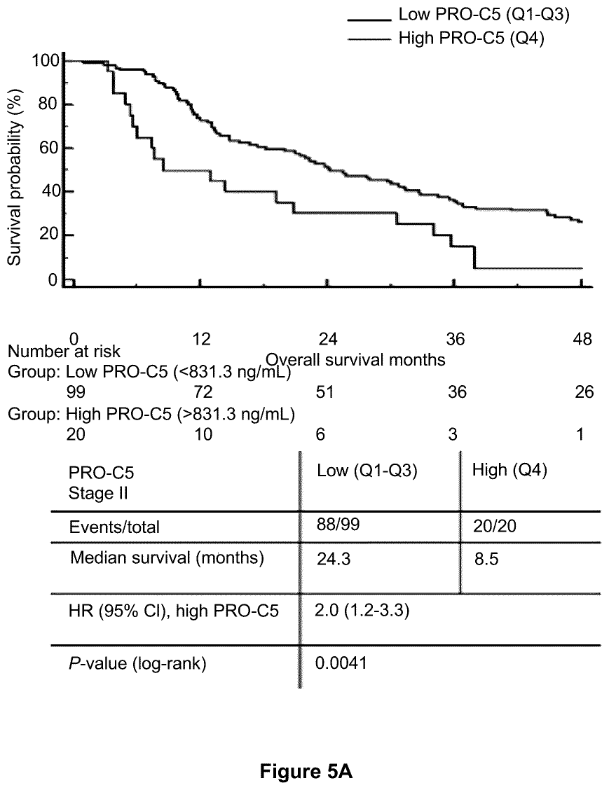

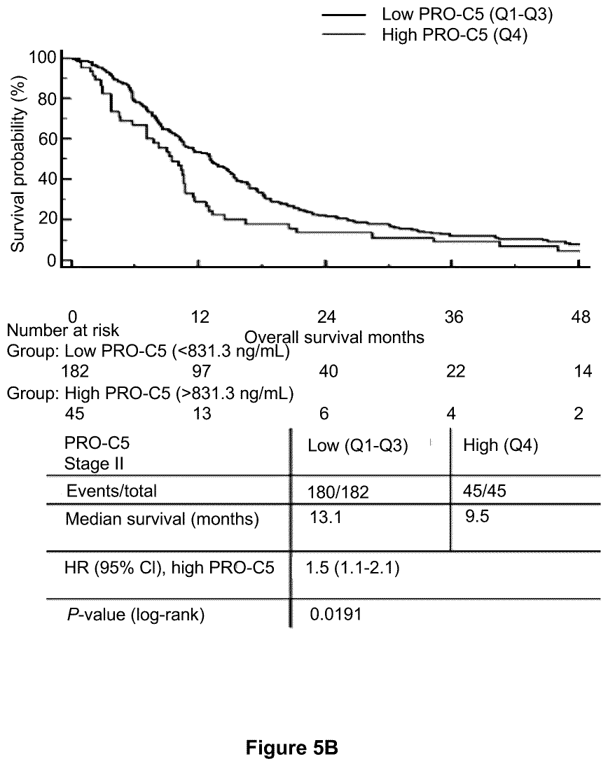

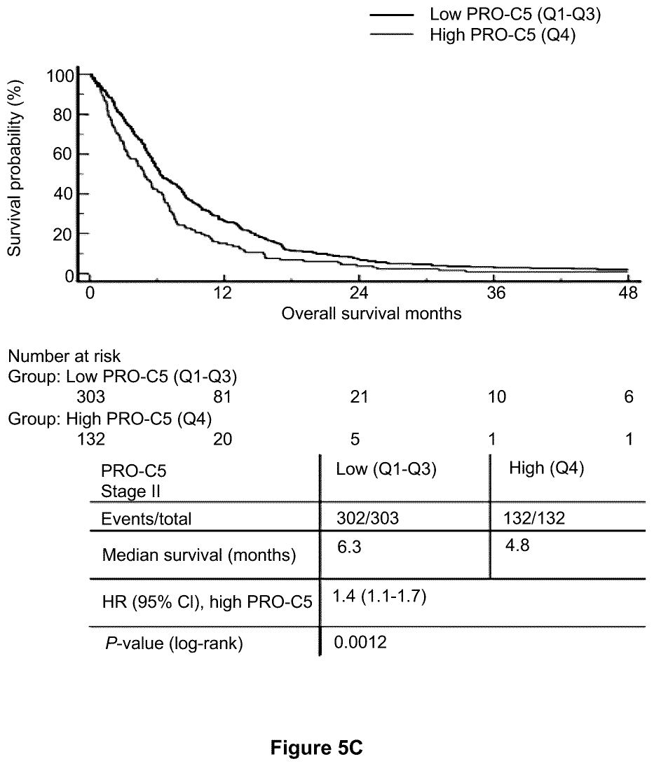

Patients in stage II (n=117), with high levels of serum PRO-C5 had a median OS of 8.5 months compared to 24.3 months for patients with low levels of PRO-C5 (log rank, p=0.0041) (FIG. 5A). Thus, the difference in median OS between low and high PRO-C5 were more than one year (15.8 months). Furthermore, univariate Cox proportional-hazard model showed that patients with stage II PDAC and high levels of PRO-C5 had 100% increased risk of mortality than patients with low levels of PRO-C5 (High PRO-C5 vs. low PRO-C5: HR 95% CI: 2.0 (1.2-3.3), p=0.0049) (FIG. 5A). In addition, a multivariate Cox proportional-hazard model including age, CA19-9, PS and CACI showed that the association between high levels of PRO-C5 and OS was independent of the clinical co-variates in patients with stage II PDAC (High PRO-C5 vs. low PRO-C5: HR 95% CI: 2.0 (1.2-3.4), p=0.0100) (Table 4). Patients with stage III PDAC (n=227) and low levels of serum PRO-C5 had a median OS at 13.1 months compared to 9.5 months for patients with high levels of serum PRO-C5 (log rank, p=0.0191) (FIG. 5B). Univariate and multivariate Cox proportional-hazard model confirmed these showing that patients with stage III PDAC and high levels of PRO-C5 had 50% increased risk of mortality than patients with low levels of PRO-C5 (High PRO-C5 vs. low PRO-C5: HR 95% CI: 1.5 (1.1-2.1), p=0.0191) (FIG. 5B). In addition, a multivariate Cox proportional-hazard model including age, CA19-9, PS and CACI showed that the association between high levels of PRO-C5 and OS was independent of the clinical co-variates in patients with stage III PDAC (High PRO-C5 vs. low PRO-C5: HR 95% CI: 1.5 (1.1-2.1), p=0.0177) (Table 4).

TABLE 4
Multivariate cox proportional-hazard regression model in
patients with PDAC divided into stage II, III and IV.

| | | Multivariate Cox proportional-hazard regression models | | | | | |
|---|---|---|---|---|---|---|---|
| | | Stage II | | Stage III | | Stage IV | |
| | | Variables | | | | | |
| | | HR | | HR | | HR | |
| | | (95% | P- | (95% | P- | (95% | P- |
| | | CI) | value | CI) | value | CI) | value |
| PRO-C5 | Q4 vs. | 2.0 | 0.0100* | 1.5 | 0.0177* | 1.3 | 0.0085* |
| | Q1-Q3 | (1.2-3.4) | | (1.1-2.1) | | (1.1-1.6) | |
| Age | Continuous | 1.00 | 0.3620 | 1.0 | 0.1825 | 1.0 | 0.5004 |
| | | (1.0-1.0) | | (1.0-.10) | | (1.0-1.0) | |
| CA19-9 | >median vs. | 1.3 | 0.1711 | 1.6 | 0.0010* | 1.3 | 0.0129* |
| | <median | (0.9-2.0) | | (1.2-2.0) | | (1.1-1.6) | |
| PS | 1 + 2 + 3 | 1.1 | 0.7235 | 1.2 | 0.1694 | 1.6 | <0.0001* |
| | vs. 0 | (0.7-1.7) | | (0.9-1.6) | | (1.3-1.9) | |
| CACI | High (≥4 | 1.2 | 0.6227 | 1.5 | 0.0577 | 1.2 | 0.0971 |
| | vs. <4) | (0.6-2.2) | | (1.0-2.3) | | (1.0-1.5) | |
| No. of | ≥1 vs. 0 | — | — | — | — | 1.0 | 0.9871 |
| metastatic | | | | | | (0.8-1.3) | |
| sites | | | | | | | |
| Liver | Yes vs. No | — | — | — | — | 1.2 | 0.1427 |
| metastasis | | | | | | (1.0-1.5) | |

Abbreviations: CACI, the Charlson age comorbidity index; CA 19-9, carcinoembryonic antigen 19-9; No, Number; and PS: performance status. Hazard ratios (HR), 95% confidence intervals (CI).
* Significant p-value.

Patients with stage IV PDAC (n=435) and low levels of serum PRO-C5 had a median OS at 6.3 months compared to 4.8 months for patients with high levels of serum PRO-C5 (log rank, p=0.0012) (FIG. 5C). A univariate Cox proportional-hazard model showed that patients with stage III PDAC and high levels of PRO-C5 had 40% increased risk of mortality than patients with low levels of PRO-C5 (High PRO-C5 vs. low PRO-C5: HR 95% CI: 1.4 (1.1-1.7), p=0.0012) (FIG. 5C). In addition, a multivariate Cox proportional-hazard model including age, CA19-9, PS, CACI, number of metastatic sites and liver metastasis showed that the association between high levels of PRO-C5 and OS was independent of the clinical co-variates in patients with stage IV PDAC (High PRO-C5 vs. low PRO-C5: HR 95% CI: 1.3 (1.1-1.6), p=0.0085) (Table 4).

The PRO-C5 Biomarker is Increased in Serum from Patients with Different Types of Cancer

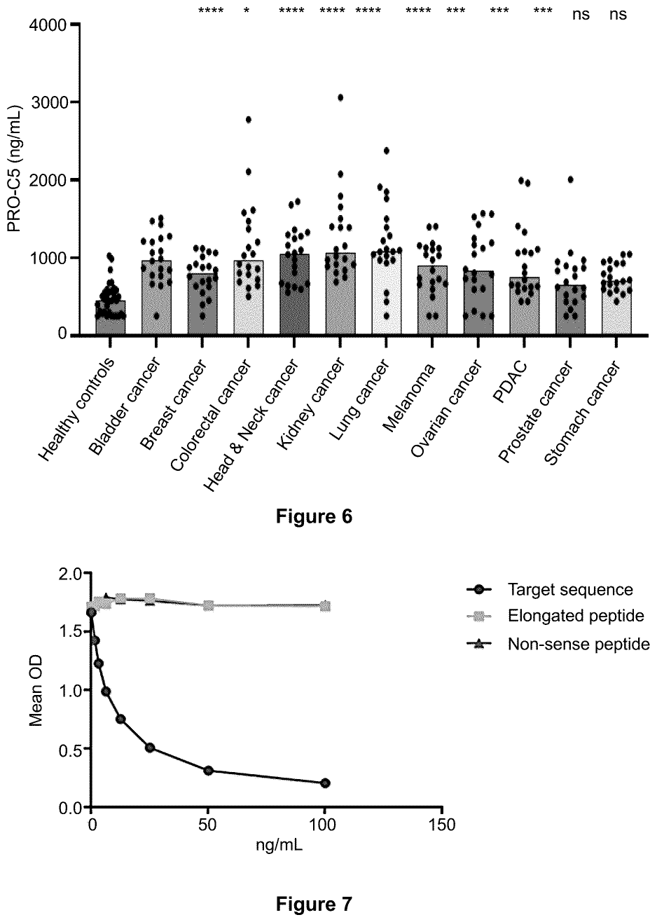

Next, the PRO-C5 biomarker potential was explored in other cancer types. Thus, PRO-C5 levels were measured in serum from patients with various stages of either bladder cancer (n=20), breast cancer (n=20), colorectal cancer (n=20), head and neck cancer (n=20), kidney cancer (n=20), lung cancer (n=20), malignant melanoma (n=20), ovarian cancer (n=20), PDAC (n=20), prostate cancer (n=20) or stomach cancer (n=20) and compared to healthy controls (n=33) (Table 2). All cancer indications had elevated PRO-C5 levels compared to healthy controls that, with the exception of prostate and stomach cancer, were statistically significant (p value ranging from <0.05-0.0001 (FIG. 6), whilst prostate and stomach cancer also showed a visual elevation in PRO-C5 levels, suggesting that PRO-C5 has biomarker potential beyond PDAC.

### DISCUSSION

The biomarker potential of measuring the α2 chain of the type V collagen pro-peptide (PRO-C5) was evaluated in serum from patients with cancer, in particular PDAC. In a discovery cohort of patients with PDAC, pancreatitis and healthy controls, PRO-C5 was increased in patients with PDAC compared to patients with pancreatitis and healthy controls. Furthermore, high levels of PRO-C5 were associated with late stage PDAC and patients with PDAC with high serum levels of PRO-C5 had increased risk of dying compared to patients with low levels. Whilst PRO-C5 has previously been associated with fibrosis in liver disease^{[63]}, this is the first time that PRO-C5 has been found to be associated with cancer.

The initial results were validated in the BIOPAC cohort where high PRO-C5 was associated with poor OS in disease stages II, III and IV independent of other risk factors including CA19-9. Interestingly, the largest relative difference in OS was seen for stage II patients where the median OS was more than one year longer in patients with low PRO-C5 compared to high PRO-C5. In comparison, CA19-9 was also independently associated with OS in patients with stage III and IV disease, but not stage II. Beyond PDAC, a relevance of PRO-C5 in other solid tumor types was shown, with a clear increase in PRO-C5 levels in multiple tumor types. The gene-expression of the α2 chain has been shown to be upregulated in many cancer indications^{[24-34]} but no studies have previously been performed on PRO-C5.

Similar to PRO-C5, some other biomarkers originating from the tumor fibrotic compartment have shown great potential as prognostic and predictive biomarkers in various cancer indications including PDAC^{[36,37,41-48]}. As an example, PRO-C3, measuring the formation of type III collagen, have shown to be prognostic for OS in patients with PDAC^{[36, 49, 50]}. Moreover, measuring the turnover of type III collagen have shown to be predictive of response to PEGPH20, an explorative anti-fibrotic drug, and was even superior to a tissue biopsy^{[46]}. Like type I collagen, type III collagen is also a major fibrillar collagen, which is present in both healthy and diseased tissue^{[51]}. In contrast to type I and type III collagen which make up the bulk of a fiber, type V collagen is more regarded as a minor collagen the assist the major fibrillar collagens in the assembly of a fiber. Lately, other minor collagens, such as the fibrillar type V and XI collagens and the FACIT collagens type XIX, XX and XXII have received an increased interest as more disease specific biomarkers and/or targets. It has been shown that biomarkers measuring type XI, XIX, XX and XXII collagens are also upregulated in various cancers^{[37,52-54]}. In addition, type XI, XX and XXII collagens were all prognostic for short OS in patients with PDAC^{[37, 52, 53]}. These minor collagens are mainly expressed in high levels during embryogenesis and in cancer progression, and their upregulation might be more pathological compared to the relative upregulation of major collagens, which are already present in high abundancy.

Interestingly, despite the fact that the major type I collagen is important for tumor progression, and has a close interaction with type V collagen, it has been shown that a biomarker measuring serum type I collagen formation, was not increased in the same cohort of different tumor types compared to controls as measured here for PRO-C5 (cohort 3). This supports a hypothesis that alterations in tumor fibrosis go beyond type I collagen. This may be because type I collagen is the most abundant protein in the body, and therefore may have less sensitivity as a serological biomarker than the less abundant minor type V collagen, also underlining the important role of type V collagen in tumor fibrosis.

Furthermore, it should be noted that not all biomarkers derived from type V collagen are suitable for measuring cancers. Levels of C5M (a type V collagen derived biomarker unrelated to PRO-C5) have been shown to be unchanged between healthy controls and patients suffering from any of bladder cancer, breast cancer, colorectal cancer, liver cancer, lung cancer, melanoma, ovarian cancer, pancreatic cancer, prostrate cancer or stomach cancer (data not shown). Therefore, it is the PRO-C5 assay specifically, that is able to discern between heathy controls and cancer patients.

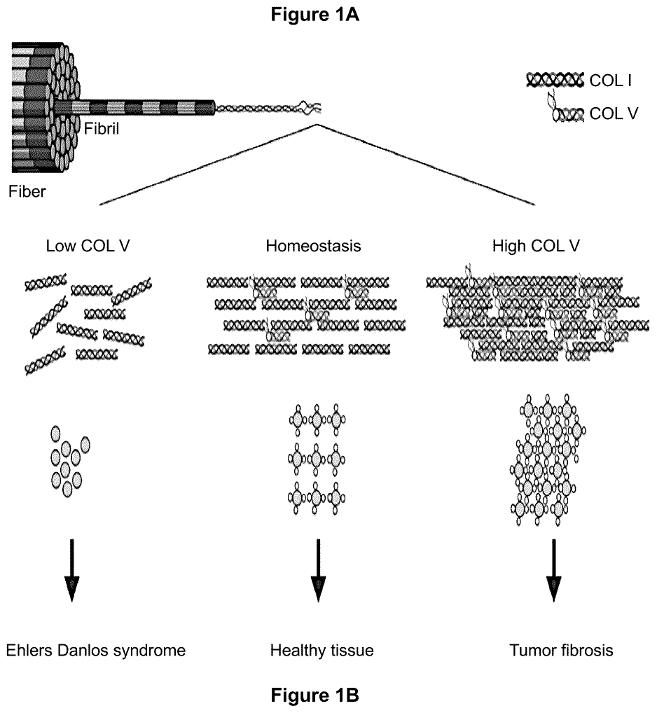

Tumor fibrosis results in stiff tissue, and it has been shown that CAFs produce more linearized and aligned collagen fibers^{[36,55-57]}. Cancer cells are thought to use these linearized collagen fibers to metastasize [58]. One study has shown that when the type V collagen/type I collagen ratio increase, in vitro, the fibrils become smaller [59, 60]. However, what this exactly means for biological function is still uncertain. Recently, Chen et al., showed that pancreatic cancer cells produce unique type I collagen homotrimers (a1/a1/a1) in comparison to the type I collagen heterotrimers (a1/a2/a1) produced by fibroblasts^{[14]}. Moreover, they showed that deletion of type I collagen homotrimers increased survival in a PDAC mouse model as well as increasing T-cell infiltration and efficacy toward anti-PD-1 immunotherapy^{[14]}. Studies have also shown that type V collagen can exist in both homo- and hetero-trimers, e.g. as α1 (V) 2α2 (V), α1 (V) 3 or α1 (V) α2 (V) α3 (V)^{[25,61,62]} but the impact of this is yet to be determined. Altogether, these data has lead the applicant to hypothesize that increased type V collagen expression leads to more linearized type I collagen fibers promoting tumor progression and metastasis, as depicted in FIG. 1. PDAC is an extremely lethal disease, and therefore it is important to treat patients as soon as possible and before they reach later stages of PDAC. PRO-C5 assays could have the potential to be used as a treatment guide already in stage II PDAC. Furthermore, as tumor fibrosis is getting more and more recognition, the interest for anti-tumor fibrosis compounds is increasing. Hence PRO-C5 assays could have the potential to monitor drug efficacy and detect responders.

In conclusion, the PRO-C5 has now been shown to be elevated in serum from cancer patients. In addition, high levels are independently associated with short OS in PDAC, especially in patients with stage II PDAC. This emphasizes the importance, and complexity, of tumor fibrosis. PRO-C5 could have the potential to be used in many aspects of drug discovery, patient stratification and drug efficacy.

### REFERENCES

1. Klein, A. P. Pancreatic Cancer: A Growing Burden. Lancet Gastroenterol. Hepatol. 2019, 4, 895-896, doi: 10.1016/S2468-1253 (19) 30323-1.
2. McGuigan, A.; Kelly, P.; Turkington, R. C.; Jones, C.; Coleman, H. G.; McCain, R. S. Pancreatic Cancer: A Review of Clinical Diagnosis, Epidemiology, Treatment and Outcomes. World J. Gastroenterol. 2018, 21, 4846-4861, doi: 10.3748/wjg.v24.143.4846.
3. Khalaf, N.; El-Serag, H. B.; Abrams, H. R.; Thrift, A. P. Burden of Pancreatic Cancer: From Epidemiology to Practice. Clin. Gastroenterol. Hepatol. 2021, 19, 876-884, doi: 10.1016/j.cgh.2020.02.054.
4. Siegel, R. L.; Miller, K. D.; Fuchs, H. E.; Jemal, A. Cancer Statistics, 2021. CA. Cancer J. Clin. 2021, 71, 7-33, doi: 10.3322/caac.21654.
5. Thomas, D.; Radhakrishnan, P. Tumor-Stromal Crosstalk in Pancreatic Cancer and Tissue Fibrosis. Mol. Cancer 2019, 18, 1-15, doi: 10.1186/s12943-018-0927-5.
6. Heldin, C. H.; Rubin, K.; Pietras, K.; Östman, A. High Interstitial Fluid Pressure—An Obstacle in Cancer Therapy. Nat. Rev. Cancer 2004, 4, 806-813, doi: 10.1038/nrc1456.
7. Mariathasan, S.; Turley, S.; Nickles, D.; Castiglioni, A.; Yuen, K.; Wang, Y.; Kadel, E. E.; Koeppen, H.; Astarita, J. L.; Cubas, R.; et al. TGFβ Attenuates Tumour Response to PD-L1 Blockade by Contributing to Exclusion of T Cells. Nature 2018, 22, 544-548.
8. Grout, J. A.; Sirven, P.; Leader, A. M.; Maskey, S.; Hector, E.; Puisieux, I.; Steffan, F.; Cheng, E.; Tung, N.; Maurin, M.; et al. Spatial Positioning and Matrix Programs of Cancer-Associated Fibroblasts Promote T Cell Exclusion in Human Lung Tumors. Cancer Discov. 2022, CD-21-1714, doi: 10.1158/2159-8290. CD-21-1714.
9. Lander, V. E.; Belle, J. I.; Kingston, N. L.; Herndon, J. M.; Hogg, G. D.; Liu, X.; Kang, L.-I.; Knolhoff, B. L.; Bogner, S. J.; Baer, J. M.; et al. Stromal Reprogramming by FAK Inhibition Overcomes Radiation Resistance to Allow for Immune Priming and Response to Checkpoint Blockade. Cancer Discov. 2022, 12, 2774-2799, doi: 10.1158/2159-8290. CD-22-0192.
10. Ogawa, Y.; Masugi, Y.; Abe, T.; Yamazaki, K.; Ueno, A.; Fujii-Nishimura, Y.; Hori, S.; Yagi, H.; Abe, Y.; Kitago, M.; et al. Three Distinct Stroma Types in Human Pancreatic Cancer Identified by Image Analysis of Fibroblast Subpopulations and Collagen. Clin. Cancer Res. 2021, 27, 107-119, doi: 10.1158/1078-0432. CCR-20-2298.
11. Chen, Y.; Kim, J.; Yang, S.; Wang, H.; Wu, C.-J.; Sugimoto, H.; LeBleu, V. S.; Kalluri, R. Type I Collagen Deletion in ASMA (+) Myofibroblasts Augments Immune Suppression and Accelerates Progression of Pancreatic Cancer. Cancer Cell 2021, 39, 548-565.e6, doi: 10.1016/j.ccell.2021.02.007.
12. Nissen, N. I.; Karsdal, M.; Willumsen, N. Collagens and Cancer Associated Fibroblasts in the Reactive Stroma and Its Relation to Cancer Biology. J. Exp. Clin. Cancer Res. 2019, 38, 115, doi: 10.1186/s13046-019-1110-6.
13. Winkler, J.; Abisoye-Ogunniyan, A.; Metcalf, K. J.; Werb, Z. Concepts of Extracellular Matrix Remodelling in Tumour

Progression and Metastasis. Nat. Commun. 2020, 11, 1-19, doi: 10.1038/s41467-020-18794-x.
14. Chen, Y.; Yang, S.; Tavormina, J.; Tampe, D.; Zeisberg, M.; Wang, H.; Mahadevan, K. K.; Wu, C. J.; Sugimoto, H.; Chang, C. C.; et al. Oncogenic Collagen I Homotrimers from Cancer Cells Bind to A3β1 Integrin and Impact Tumor Microbiome and Immunity to Promote Pancreatic Cancer. Cancer Cell 2022, 40, 818-834.e9, doi: 10.1016/j.ccell.2022.06.011.
15. Armstrong, T.; Packham, G.; Murphy, L. B.; Bateman, A. C.; Conti, J. A.; Fine, D. R.; Johnson, C. D.; Benyon, R. C.; Iredale, J. P. Type I Collagen Promotes the Malignant Phenotype of Pancreatic Ductal Adenocarcinoma. Clin. Cancer Res. 2004, 10, 7427-7437.
16. Menke, A.; Philippi, C.; Vogelmann, R.; Seidel, B.; Lutz, M. P.; Adler, G.; Wedlich, D. Down-Regulation of E-Cadherin Gene Expression by Collagen Type I and Type III in Pancreatic Cancer Cell Lines 1. Biochemistry 2001, 61, 3508-3517.
17. Cheng, J. C.; Leung, P. C. K. Type I Collagen Down-Regulates E-Cadherin Expression by Increasing PI3KCA in Cancer Cells. Cancer Lett. 2011, doi: 10.1016/j.canlet.2011.02.008.
18. Gao, H.; Chakraborty, G.; Zhang, Z.; Akalay, I.; Gadiya, M.; Gao, Y.; Sinha, S.; Hu, J.; Jiang, C.; Akram, M.; et al. Multi-Organ Site Metastatic Reactivation Mediated by Non-Canonical Discoidin Domain Receptor 1 Signaling. Cell 2016, 166, 47-62, doi: 10.1016/j.cell.2016.06.009.
19. Barcus, C. E.; O'Leary, K. A.; Brockman, J. L.; Rugowski, D. E.; Liu, Y.; Garcia, N.; Yu, M.; Keely, P. J.; Eliceiri, K. W.; Schuler, L. A. Elevated Collagen-I Augments Tumor Progressive Signals, Intravasation and Metastasis of Prolactin-Induced Estrogen Receptor Alpha Positive Mammary Tumor Cells. Breast Cancer Res. 2017, 19, 1-13, doi: 10.1186/s13058-017-0801-1.
20. Su, H.; Yang, F.; Fu, R.; Trinh, B.; Sun, N.; Liu, J.; Kumar, A.; Baglieri, J.; Siruno, J.; Le, M.; et al. Collagenolysis-Dependent DDR1 Signalling Dictates Pancreatic Cancer Outcome. Nature 2022, 610, 366-372, doi: 10.1038/s41586-022-05169-z.
21. Symoens, S.; Renard, M.; Bonod-Bidaud, C.; Syx, D.; Vaganay, E.; Malfait, F.; Ricard-Blum, S.; Kessler, E.; Van Laer, L.; Coucke, P.; et al. Identification of Binding Partners Interacting with the A1-N-Propeptide of Type V Collagen. Biochem. J. 2010, 433, 371-381, doi: 10.1042/BJ20101061.
22. Wenstrup, R. J.; Florer, J. B.; Brunskill, E. W.; Bell, S. M.; Chervoneva, I.; Birk, D. E. Type V Collagen Controls the Initiation of Collagen Fibril Assembly. J. Biol. Chem. 2004, 279, 53331-53337, doi: 10.1074/jbc.M409622200.
23. Aszódi, A.; Legate, K. R.; Nakchbandi, I.; Fässler, R. What Mouse Mutants Teach Us about Extracellular Matrix Function. Annu. Rev. Cell Dev. Biol. 2006, 22, 591-621, doi: 10.1146/annurev.cellbio.22.010305.104258.
24. Sun, M.; Chen, S.; Adams, S. M.; Florer, J. B.; Liu, H.; Kao, W. W. Y.; Wenstrup, R. J.; Birk, D. E. Collagen V Is a Dominant Regulator of Collagen Fibrillogenesis: Dysfunctional Regulation of Structure and Function in a Corneal-Stroma-Specific Col5α1-Null Mouse Model. J. Cell Sci. 2011, 124, 4096-4105, doi: 10.1242/jcs.091363.
25. Mak, K. M.; Png, C. Y. M.; Lee, D. J. Type V Collagen in Health, Disease, and Fibrosis. Anat. Rec. 2016, 299, 613-629, doi: 10.1002/ar.23330.
26. Tan, Y.; Chen, Q. The TCGA and GEO Databases Identify COL5A2 as a Poorly Prognostic Gene in Patients with Advanced Gastric Cancer. Res. Sq. 2018, 1-19.
27. Wang, J.; Jiang, Y. H.; Yang, P. Y.; Liu, F. Increased Collagen Type v A2 (Col5α2) in Colorectal Cancer Is Associated with Poor Prognosis and Tumor Progression. Onco. Targets. Ther. 2021, 14, 2991-3002.
28. Tan, Y.; Chen, Q.; Xing, Y.; Zhang, C.; Pan, S.; An, W.; Xu, H. High Expression of COL5A2, a Member of COL5 Family, Indicates the Poor Survival and Facilitates Cell Migration in Gastric Cancer. Biosci. Rep. 2021, 41, 1-15, doi: 10.1042/BSR20204293.
29. Barsky, S. H.; Rao, C. N.; Grotendorst, G. R.; Liotta, L. A. Increased Content of Type V Collagen in Desmoplasia of Human Breast Carcinoma. Am. J. Pathol. 1982, 108, 276-283.
30. Berchtold, S.; Grünwald, B.; Krüger, A.; Reithmeier, A.; Hähl, T.; Cheng, T.; Feuchtinger, A.; Born, D.; Erkan, M.; Kleeff, J.; et al. Collagen Type V Promotes the Malignant Phenotype of Pancreatic Ductal Adenocarcinoma. Cancer Lett. 2015, 356, 721-732, doi: 10.1016/j.canlet.2014.10.020.
31. Imamura, T.; Iguchi, H.; Manabe, T.; Ohshio, G.; Yoshimura, T.; Wang, Z. H.; Suwa, H.; Ishigami, S.; Imamura, M. Quantitative Analysis of Collagen and Collagen Subtypes I, III, and V in Human Pancreatic Cancer, Tumor-Associated Chronic Pancreatitis, and Alcoholic Chronic Pancreatitis. Pancreas 1995, 11, 357-364.
32. Huang, G.; Ge, G.; Izzi, V.; Greenspan, D. S. A3 Chains of Type v Collagen Regulate Breast Tumour Growth via Glypican-1. Nat. Commun. 2017, 8, 1-17, doi: 10.1038/ncomms14351.
33. Balancin, M. L.; Teodoro, W. R.; Baldavira, C. M.; Prieto, T. G.; Farhat, C.; Velosa, A. P.; da Costa Souza, P.; Yaegashi, L. B.; Ab'Saber, A. M.; Takagaki, T. Y.; et al. Different Histological Patterns of Type-V Collagen Levels Confer a Matrices-Privileged Tissue Microenvironment for Invasion in Malignant Tumors with Prognostic Value. Pathol. Res. Pract.

2020, 216, 153277, doi: 10.1016/j.prp.2020.153277.
34. Ren, W.; Zhang, Y.; Zhang, L.; Lin, Q.; Zhang, J.; Xu, G. Overexpression of Collagen Type V A1 Chain in Human Breast Invasive Ductal Carcinoma Is Mediated by TGF-B1. Int. J. Oncol. 2018, 52, 1694-1704.
35. Chen, I.; Jensen, B. V.; Bojesen, S. E.; Johansen, A. Z.; Schultz, N. A.; Hansen, C. P.; Hasselby, J. P.; Holländer, N. H.; Nissen, M. H. B.; Bjerregaard, J. K.; et al. Identification of New Biomarkers in Patients with Pancreatic Cancer (BIOPAC): A Study Protocol of an Open Cohort Study. J. Cancer Sci. Ther. 2019, 11, 232.
36. Nissen, N. I.; Johansen, A. Z.; Chen, I. M.; Johansen, J. S.; Pedersen, R. S.; Hansen, C. P.; Karsdal, M.; Willumsen, N. Collagen Biomarkers Quantify Fibroblast Activity In Vitro and Predict Survival in Patients with Pancreatic Ductal Adenocarcinoma. Cancers (Basel). 2022, 14, 1-21, doi: 10.3390/cancers14030819.
37. Nissen, N. I.; Kehlet, S.; Johansen, A. Z.; Chen, I. M.; Karsdal, M.; Johansen, J. S.; Diab, H. M. H.; Jørgensen, L. N.; Sun, S.; Manon-Jensen, T.; et al. Noninvasive Prognostic Biomarker Potential of Quantifying the Propeptides of Type XI Collagen Alpha-1 Chain (PRO-C11) in Patients with Pancreatic Ductal Adenocarcinoma. Int. J. Cancer 2021, 149, 228-238, doi: 10.1002/ijc.33551.
38. Vassiliadis, E.; Veidal, S. S.; Hansen, C.; Karsdal, M. A.; Leeming, D. J. Circulating Levels of a Collagen Type V Propeptide Fragment in a Carbon Tetrachloride Reversible Model of Liver Fibrosis. Biomark. Insights 2012, 7, 159-166, doi: 10.4137/BMI. S10975.
39. Vassiliadis, E.; Veidal, S. S.; Simonsen, H.; Larsen, D. V.; Vainer, B.; Chen, X.; Zheng, Q.; Karsdal, M. A.; Leeming, D. J. Immunological Detection of the Type v Collagen Propeptide Fragment, PVCP-1230, in Connective Tissue Remodeling Associated with Liver Fibrosis. Biomarkers 2011, doi: 10.3109/1354750x.2011.584131.
40. Altman, D. G.; McShane, L. M.; Sauerbrei, W.; Taube, S. E. Reporting Recommendations for Tumor Marker Prognostic Studies (REMARK): Explanation and Elaboration. PLOS Med. 2012, 9, doi: 10.1371/journal.pmed.1001216.
41. Nissen, N. I.; Kehlet, S.; Boisen, M. K.; Liljefors, M.; Jensen, C.; Johansen, A. Z.; Johansen, J. S.; Erler, J. T.; Karsdal, M.; Mortensen, J. H.; et al. Prognostic Value of Blood-Based Fibrosis Biomarkers in Patients with Metastatic Colorectal Cancer Receiving Chemotherapy and Bevacizumab. Sci. Rep. 2021, 11, 1-12, doi: 10.1038/s41598-020-79608-0.
42. SN, K.; A, H.; MK, B.; J S, J.; MA, K.; N, W.; J, E. Prognostic Evaluation of a New Class of Liquid Biopsy Biomarkers in Patients with Metastatic Colorectal Cancer: Using the Tumor Microenvironment as a Source of Protein Biomarkers. J. Clin. Oncol. 2018, 15, 3588-3588, doi: DOI: 10.1200/JCO.2018.36.15 suppl.3588.
43. Willumsen, N.; Thomsen, L. B.; Bager, C. L.; Jensen, C.; Karsdal, M. A. Quantification of Altered Tissue Turnover in a Liquid Biopsy: A Proposed Precision Medicine Tool to Assess Chronic Inflammation and Desmoplasia Associated with a pro-Cancerous Niche and Response to Immuno-Therapeutic Anti-Tumor Modalities. Cancer Immunol. Immunother. 2018, 67, doi: 10.1007/s00262-017-2074-z.
44. Jensen, C.; Nissen, N. I.; Von Arenstorff, C. S.; Karsdal, M. A.; Willumsen, N. Serological Assessment of Collagen Fragments and Tumor Fibrosis May Guide Immune Checkpoint Inhibitor Therapy. J. Exp. Clin. Cancer Res. 2021, 40, 1-11, doi: 10.1186/s13046-021-02133-z.
45. Willumsen, N.; Jensen, C.; Green, G.; Nissen, N. I.; Neely, J.; Nelson, D. M.; Pedersen, R. S.; Frederiksen, P.; Chen, I. M.; Boisen, M. K.; et al. Fibrotic Activity Quantified in Serum by Measurements of Type III Collagen Pro-Peptides Can Be Used for Prognosis across Different Solid Tumor Types. Cell. Mol. Life Sci. 2022 794 2022, 79, 1-11, doi: 10.1007/S00018-022-04226-0.
46. Wang, S.; Bager, C. L.; Karsdal, M. A.; Chondros, D.; Taverna, D.; Willumsen, N. Blood-Based Extracellular Matrix Biomarkers as Predictors of Survival in Patients with Metastatic Pancreatic Ductal Adenocarcinoma Receiving Pegvorhyaluronidase Alfa. J. Transl. Med. 2021, 19, 1-12.
47. Jensen, C.; Holm Nielsen, S.; Eslam, M.; Genovese, F.; Nielsen, M. J.; Vongsuvanh, R.; Uchila, R.; van der Poorten, D.; George, J.; Karsdal, M. A.; et al. <p>Cross-Linked Multimeric Pro-Peptides of Type III Collagen (PC3X) in Hepatocellular Carcinoma-A Biomarker That Provides Additional Prognostic Value in AFP Positive Patients</P>. J. Hepatocell. Carcinoma 2020, Volume 7, 301-313, doi: 10.2147/jhc.s275008.
48. Jensen, C.; Sinkeviciute, D.; Madsen, D. H.; Önnerfjord, P.; Hansen, M.; Schmidt, H.; Karsdal, M. A.; Svane, I. M.; Willumsen, N. Granzyme B Degraded Type IV Collagen Products in Serum Identify Melanoma Patients Responding to Immune Checkpoint Blockade. Cancers (Basel). 2020, 12, E2786, doi: 10.3390/cancers12102786.
49. Chen, I. M.; Willumsen, N.; Dehlendorff, C.; Johansen, A. Z.; Jensen, B. V.; Hansen, C. P.; Hasselby, J. P.; Bojesen, S. E.; Pfeiffer, P.; Nielsen, S. E.; et al. Clinical Value of Serum Hyaluronan and Propeptide of Type III Collagen in Patients with Pancreatic Cancer. Int. J. Cancer 2020, 146, 2913-2922, doi: 10.1002/ijc.32751.
50. Willumsen, N.; Ali, S. M.; Leitzel, K.; Drabick, J. J.; Yee, N.; Polimera, H. V.; Nagabhairu, V.; Krecko, L.; Ali, A.; Maddukuri, A.; et al. Collagen Fragments Quantified in Serum as Measures of Desmoplasia Associate with Survival Outcome in Patients with Advanced Pancreatic Cancer. Sci. Rep. 2019, 9, 19761, doi: 10.1038/s41598-019-56268-3.
51. Ricard-Blum, S. The Collagen Family. Cold Spring Harb. Perspect. Biol. 2011, 3, a004978-a004978, doi: 10.1101/cshperspect. a004978.
52. Madsen, E. A.; Thorlacius-Ussing, J.; Nissen, N. I.; Jensen, C.; Chen, I. M.; Johansen, J. S.; Diab, H. M. H.; Jørgensen, L. N.; Hansen, C. P.; Karsdal, M. A.; et al. Type XXII Collagen Complements Fibrillar Collagens in the Serological Assessment of Tumor Fibrosis and the Outcome in Pancreatic Cancer. Cells 2022, 11.
53. Thorlacius-Ussing, J.; Jensen, C.; Madsen, E. A.; Nissen, N. I.; Manon-Jensen, T.; Chen, I. M.; Johansen, J. S.; Diab, H. M. H.; Jørgensen, L. N.; Karsdal, M. A.; et al. Type XX Collagen Is Elevated in Circulation of Patients with Solid Tumors. Int. J. Mol. Sci. 2022, 23, 4144, doi: 10.3390/IJMS23084144.
54. Thorlacius-Ussing, J.; Manon-Jensen, T.; Sun, S.; Leeming, D. J.; Sand, J. M.; Karsdal, M.; Willumsen, N. Serum Type Xix Collagen Is Significantly Elevated in Non-Small Cell Lung Cancer: A Preliminary Study on Biomarker Potential. Cancers (Basel). 2020, 12, 1-14, doi: 10.3390/cancers12061510.
55. Drifka, C. R.; Loeffler, A. G.; Mathewson, K.; Keikhosravi, A.; Eickhoff, J. C.; Liu, Y.; Weber, S. M.; John Kao, W.; Eliceiri, K. W. Highly Aligned Stromal Collagen Is a Negative Prognostic Factor Following Pancreatic Ductal Adenocarcinoma Resection. Oncotarget 2016, 7, 76197-76213, doi: 10.18632/oncotarget.12772.
56. Bolm, L.; Zghurskyi, P.; Lapshyn, H.; Petrova, E.; Zemskov, S.; Vashist, Y. K.; Deichmann, S.; Honselmann, K. C.; Bronsert, P.; Keck, T.; et al. Alignment of Stroma Fibers, Microvessel Density and Immune Cell Populations Determine Overall Survival in Pancreatic Cancer—An Analysis of Stromal Morphology. PLOS One 2020, 15, e0234568-e0234568, doi: 10.1371/journal.pone. 0234568.
57. Drifka, C. R.; Tod, J.; Loeffler, A. G.; Liu, Y.; Thomas, G. J.; Eliceiri, K. W.; Kao, W. J. Periductal Stromal Collagen Topology of Pancreatic Ductal Adenocarcinoma Differs from That of Normal and Chronic Pancreatitis. Mod. Pathol. 2015, 28, 1470-1480, doi: 10.1038/modpathol.2015.97.
58. Drifka, C. R.; Loeffler, A. G.; Esquibel, C. R.; Weber, S. M.; Eliceiri, K. W.; Kao, W. J. Human Pancreatic Stellate Cells Modulate 3D Collagen Alignment to Promote the Migration of Pancreatic Ductal Adenocarcinoma Cells. Biomed. Microdevices 2016, 18, 105, doi: 10.1007/s10544-016-0128-1.
59. Birk, D. E.; Fitch, J. M.; Babiarz, J. P.; Doane, K. J.; Linsenmayer, T. F. Collagen Fibrillogenesis in Vitro: Interaction of Types I and V Collagen Regulates Fibril Diameter. J. Cell Sci. 1990, 95, 649-657, doi: 10.1242/jcs.95.4.649.
60. Adachi, E.; Hayashi, T. In Vitro Formation of Hybrid Fibrils of Type v Collagen and Type I Collagen Limited Growth of Type I Collagen Into Thick Fibrils by Type V Collagen. Connect. Tissue Res. 1986, 14, 257-266, doi: 10.3109/03008208609017469.
61. Chanut-Delalande, H.; Fichard, A.; Bernocco, S.; Garrone, R.; Hulmes, D. J. S.; Ruggiero, F. Control of Heterotypic Fibril Formation by Collagen V Is Determined by Chain Stoichiometry*. J. Biol. Chem. 2001, 276, 24352-24359, doi: https://doi.org/10.1074/jbc.M101182200.
62. Chanut-Delalande, H.; Bonod-Bidaud, C.; Cogne, S.; Malbouyres, M.; Ramirez, F.; Fichard, A.; Ruggiero, F. Development of a Functional Skin Matrix Requires Deposition of Collagen V Heterotrimers. Mol. Cell. Biol. 2004, 24, 6049-6057, doi: 10.1128/MCB.24.13.6049-6057.2004.
63. Leeming, D. J.; Veidal, S. S.; Karsdal, M. A.; Nielsen, M. J.; Trebicka, J.; Busk, T.; Bendtsen, F.; Krag, A.; Møller, S. Pro-C5, a Marker of True Type V Collagen Formation and Fibrillation, Correlates with Portal Hypertension in Patients with Alcoholic Cirrhosis. Scand. J. Gastroenterol. 2015, 50, 584-592, doi: 10.3109/00365521.2014.996590.

1: A method of immunoassay for detecting and/or monitoring a cancer in a patient, the method comprising;
i) contacting a patient sample with a monoclonal antibody that specifically binds to the N-terminus amino acid sequence TAALGDIMGH (SEQ ID NO: 1);
ii) detecting and determining the amount of binding between said monoclonal antibody and peptides in the sample; and
ii) correlating said amount of binding with values associated with normal healthy subjects and/or values associated with known disease severity and/or values obtained from said patient at a previous time point and/or with a predetermined cut-off value.

2: The method of claim 1, wherein the method is a method of immunoassay for detecting and/or monitoring a bladder cancer, breast cancer, colorectal cancer, head and neck cancer, kidney cancer, lung cancer, melanoma, ovarian cancer, pancreatic cancer, prostate cancer or stomach cancer.

3: The method of claim 1, wherein the method is a method of immunoassay for detecting and/or monitoring pancreatic ductal adenocarcinoma.

4: The method of claim 1, wherein the method is a method of immunoassay for detecting a cancer of a particular severity in a patient.

5: The method of claim 4, wherein the method is a method for detecting a stage of a cancer in a patient.

6: The method of claim 4, wherein the method is a method for predicting a level of severity associated with a likely or average overall survival time.

7: The method of claim 1, wherein the monoclonal antibody does not specifically bind to a peptide having the N-terminus amino acid sequence LTAALGDIMGH (SEQ ID NO: 2).

8: The method of claim 1, wherein the monoclonal antibody does not specifically bind to a peptide having the N-terminus amino acid sequence AALGDIMGH (SEQ ID NO: 3).

9: The method of claim 1, wherein the monoclonal antibody is raised against a synthetic peptide having the N-terminus amino acid sequence TAALGDIMGH (SEQ ID NO: 1).

10: The method of claim 1, wherein the patient sample is selected from blood, serum or plasma.

11: The method of claim 1, wherein the immunoassay is a competition assay or a sandwich assay.

12: The method of claim 1, wherein the immunoassay is a radio-immunoassay or an enzyme-linked immunosorbent assay.


SEQUENCE LISTING (5 sequences)

SEQ ID NO: 1 - protein, 10 aa, synthetic construct
TAALGDIMGH

SEQ ID NO: 2 - protein, 11 aa, synthetic construct
LTAALGDIMGH

SEQ ID NO: 3 - protein, 9 aa, synthetic construct
AALGDIMGH

SEQ ID NO: 4 - protein, 13 aa, synthetic construct
  SITE 13: note = linked to ovalbumin
TAALGDIMGHGGC

SEQ ID NO: 5 - protein, 13 aa, synthetic construct
  SITE 13: note = biotinylated
TAALGDIMGHGGC